(19) United States
(12) Patent Application Publication
(10) Pub. No.: US 2023/0278943 A1
(43) Pub. Date: Sep. 7, 2023

(54) METHOD FOR PRODUCING POLYOXYMETHYLENE DIMETHYL ETHERS

(71) Applicant: FRAUNHOFER-GESELLSCHAFT ZUR FÖRDERUNG DER ANGEWANDTEN FORSCHUNG E.V., München (DE)

(72) Inventors: Franz Mantei, Freiburg (DE); Mohamed Ouda, Freiburg (DE); Achim Schaadt, Freiburg (DE)

(21) Appl. No.: 18/015,459

(22) PCT Filed: Jul. 12, 2021
(86) PCT No.: PCT/EP2021/069282

§ 371 Date: Jan. 10, 2023

(30) Foreign Application Priority Data
Jul. 13, 2020 (DE) 10 2020 118 386.0

(51) Int. Cl. C07C 41/56; C07C 41/58; C08G 65/00

(52) U.S. Cl. CPC C07C 41/56; C07C 41/58; C08G 65/00

(57) ABSTRACT

The present invention relates to a process for preparing polyoxymethylene dimethyl ethers, comprising the following steps:
reaction of formaldehyde and methylal (OME₁) in a reactor R1 to obtain a product mixture,
distillative separation of the product mixture in a distillation unit D1 into a top stream which contains OME₁, OME₂, formaldehyde, methanol and water, and a bottom stream which contains OME_{≥3},
mixing of the top stream drawn off from the distillation unit D1 with a methanol-containing stream,
treatment of the mixture in a reactive distillation unit RD2 to form a top stream containing methylal and a water-containing bottom stream,
introduction of the bottom stream drawn off from the distillation unit D1 into a distillation unit D3 and distillative separation of the polyoxymethylene dimethyl ethers.

The present invention relates to a process for preparing polyoxymethylene dimethyl ethers.

Synthetic energy carriers that are not produced on the basis of crude oil or natural gas can reduce dependence on fossil energy sources and the environmental pollution resulting from their use. One example of such an energy carrier are polyoxymethylene dimethyl ethers (OMEs). Polyoxymethylene dimethyl ethers (OMEs) can be prepared from carbon dioxide and water and, if renewable energy carriers are used for their production, have the potential to close the carbon dioxide cycle when burnt as fuel.

In addition, the use of polyoxymethylene dimethyl ethers as an energy carrier offers further advantages. Polyoxymethylene dimethyl ethers have no carbon-carbon bonds and also have a high oxygen content. Polyoxymethylene dimethyl ethers burn soot-free and are thus gentle both on the combustion engine and downstream filter elements and also on the environment. In addition, soot-free combustion enables a reduction in nitrogen oxide emissions. Polyoxymethylene dimethyl ethers having three to five oxymethylene units (OME₃₋₅) are of particular interest because of their diesel-like properties.

The reactants typically used in the synthesis of polyoxymethylene dimethyl ethers are a formaldehyde source (e.g. formaldehyde, trioxane, or paraformaldehyde) and a compound for methyl capping such as methanol, methylal or dimethyl ether. If the reactant mixture contains methanol and water, these react with formaldehyde to give polyoxymethylene glycols (MGₙ; HO—(CH₂O)ₙ—H) and polyoxymethylene hemiacetals (HFₙ; HO—(CH₂O)ₙ—CH₃) according to the following reaction equations 1-4. These reactions do not require the presence of a catalyst and reach chemical equilibrium very rapidly. In addition, the chemical equilibrium lies very predominantly on the product side, meaning that essentially no monomeric formaldehyde (CH₂O) is present in the product mixture. The formation of methylal (H₃C—O—(CH₂O)₁—CH₃; OME₁) via the acetalization reaction between methanol and HO—CH₂O—CH₃ (HF₁) according to the following reaction equation 5 requires the presence of an acidic catalyst. Chain growth is achieved by the incorporation of further CH₂O units according to the following reaction equation 7. Further acetalization reactions between methanol and the polyoxymethylene hemiacetals HFₙ take place according to the following reaction equation 6. Possible side reactions to form trioxane ((CH₂O)₃) and methyl formate (HC(O)OCH₃) are shown by the following reaction equations 8 and 9.

| | | | |
|---|---|---|---|
| CH₂O + H₃C—OH | ⇄ | HO—(CH₂O)₁—CH₃ | 1 |
| CH₂O + HO—(CH₂O)ₙ₋₁—CH₃ | ⇄ | HO—(CH₂O)ₙ—CH₃, n ≥ 2 | 2 |
| CH₂O + H₂O | ⇄ | HO—(CH₂O)₁—H | 3 |
| CH₂O + HO—(CH₂O)ₙ₋₁—H | ⇄ | HO—(CH₂O)ₙ—H, n ≥ 2 | 4 |
| H₃C—OH + HO—(CH₂O)₁—CH₃ | ⇄^{H+} | H₃C—O—(CH₂O)₁—CH₃ + H₂O | 5 |
| H₃C—OH + HO—(CH₂O)ₙ—CH₃ | ⇄^{H+} | H₃C—O—(CH₂O)ₙ—CH₃ + H₂O | 6 |
| CH₂O + H₃C—O—(CH₂O)ₙ₋₁—CH₃ | ⇄^{H+} | H₃C—O—(CH₂O)ₙ—CH₃, n ≥ 2 | 7 |
| 3 CH₂O | ⇄^{H+} | (CH₂O)₃ | 8 |
| 2 CH₂O | → | HCOOCH₃ | 9 |

An overview of known preparation processes for polyoxymethylene dimethyl ethers H₃C—O—(CH₂O)ₙ—CH₃ with n≥2 (OME_{≥2}), in particular n=3-5 (OME₃₋₅), can be found for example in the publication by M. Ouda et al., React. Chem. Eng., 2017, 2, pp. 50-59. A distinction is made here between anhydrous and aqueous synthesis routes. Anhydrous synthesis routes exhibit a reduced formation of by-products, but provision of the reactants is energy-intensive. The provision of the reactants for aqueous synthesis routes is less energy-intensive, but further by-products and water are present in the reaction product.

By way of example, a reactant mixture containing methylal and trioxane may be used as a starting point. The advantage with this synthesis variant is that it produces the polyoxymethylene dimethyl ethers in a high yield and can be conducted essentially in the absence of water, which reduces the number of by-products. The disadvantage is that the preparation of anhydrous trioxane is very energy-intensive and complex, which adversely affects the energy efficiency and the economic feasibility of the process.

US 2007/260094 A1 describes a process for preparing polyoxymethylene dimethyl ethers in which methylal and trioxane are fed into a reactor and are reacted in the presence of an acidic catalyst, with the amount of water introduced into the reaction mixture being less than 1% by weight.

An essentially anhydrous synthesis of polyoxymethylene dialkyl ethers is also enabled by the use of trioxane and a dialkyl ether (such as for example dimethyl ether DME) as reactants.

DE 10 2005 027690 A1 describes a process for preparing polyoxymethylene dialkyl ethers in which a dialkyl ether (dimethyl ether, methyl ethyl ether or diethyl ether) and trioxane are fed into a reactor and are reacted in the presence of an acidic catalyst, with the amount of water introduced into the reaction mixture by the dialkyl ether, trioxane and/or the catalyst being less than 1% by weight. P. Haltenort et al., Catalysis Communications, 2018, 109, 80, describe the synthesis of polyoxymethylene dimethyl ethers from dimethyl ether (DME) and trioxane using a zeolite as acidic catalyst. The maximum DME conversion was 13.9% by weight and the maximum yield of OME₃-s, based on the reactants used, was 8.2% by weight. Investigations have shown that the synthesis variant proceeding from dimethyl ether and trioxane leads to relatively high reactor residence times.

It is also known to prepare the formaldehyde used for the polyoxymethylene dimethyl ether synthesis via a catalytic dehydrogenation of methanol, see for example M. Ouda, F. Mantei et al., React. Chem. Eng., 2018, 129, 11164. This approach enables the use of an anhydrous reactant mixture, meaning that only the water formed during the synthesis is present in the product mixture. However, the catalytic dehydrogenation of methanol is a complex chemical reaction for which the degree of technological maturity is still relatively low.

Also known is the use of an aqueous formaldehyde- and methanol-containing reactant mixture.

DE 10 2016 222657 A1 describes a process for preparing polyoxymethylene dimethyl ethers, comprising the following steps:
(i) feeding of formaldehyde, methanol and water into a reactor R and reaction to form a reaction mixture containing formaldehyde, water, methylene glycol, polyoxymethylene glycols, methanol, hemiformals, methylal and polyoxymethylene dimethyl ethers;
(ii) feeding of the reaction mixture into a reactive distillation column K1 and separation into a low boiler fraction F1 containing formaldehyde, water, methylene glycol, polyoxymethylene glycols, methanol, hemiformals, methylal and polyoxymethylene dimethyl ethers having 2 to 3 oxymethylene units (OME₂₋₃), and a high boiler fraction F2 containing polyoxymethylene dimethyl ethers having more than two oxymethylene units (OME_{≥3}).

Less energy is expended to prepare an aqueous formaldehyde solution compared to an anhydrous formaldehyde source such as trioxane. However, when using reactants in aqueous phase, there is the challenge of removing the water from the product mixture as efficiently as possible. In addition, the presence of water leads to the formation of further by-products, which reduces the product yield.

The disadvantage of using methanol as reactant is that the acetalization reaction between methanol and the hemiacetals leads to additional water being formed as by-product (see the above reaction equations 5 and 6) and this water of reaction also needs to be removed from the process. Due to the complex distillation behavior of the product mixture, the water cannot be removed by a standard distillation without also removing some of the formaldehyde at the same time. As an alternative to distillative removal, other methods for removing water have therefore also been investigated, such as for example adsorption, membrane-based separation processes or extraction. However, these methods exhibit further challenges for long-term operation. These alternative water removal methods are described for example in the following publications:
N. Schmitz et al., Industrial & Engineering Chemistry Research, 2017, 56, 11519;
N. Schmitz et al., Journal of Membrane Science, 2018, 564, 806;
L. Wang et al., J. Chem. Eng. Data, 2018, 63, 3074;
M. Shi et al., Can. J. Chem. Eng., 2018, 96, 968;
D. Oestreich et al., Fuel, 2018, 214, 39;
X. Li et al., J. Chem. Eng. Data, 2019, 64, 5548.

Methylal is used as a solvent and in the production of perfume, resins or protective coatings. It is also being tested as a fuel additive and as a synthetic fuel. The preparation of very substantially pure methylal is described for example in WO 2012/062822 A1. In this process, formaldehyde and methanol react to give a product mixture containing methylal and water and also unconverted methanol and formaldehyde. The product mixture is separated into three fractions in a reactive distillation unit. The fraction which leaves the reactive distillation unit as top stream is rich in methylal. The preparation of polyoxymethylene dimethyl ethers is not described.

One object of the present invention is that of preparing polyoxymethylene dimethyl ethers via an efficient and readily scalable process.

The object is achieved by the two alternative processes according to the invention that are described below (“first independent embodiment” and “second independent embodiment”).

According to a first independent embodiment of the present invention, the object is achieved by a process for preparing polyoxymethylene dimethyl ethers, comprising the following steps:
mixing of a stream S_{FA} containing a water-containing formaldehyde source with a stream S_{OME1} containing methylal, to obtain a reactant mixture M_{Reactant},
reaction of the reactant mixture M_{Reactant} in a reactor R1 to obtain a product mixture M_{R1} containing polyoxymethylene dimethyl ethers of the formulae H₃C—O—(CH₂O)₂—CH₃ (OME₂), H₃C—O—(CH₂O)₃₋₅—CH₃ (OME₃₋₅) and H₃C—O—(CH₂O)ₙ—CH₃ with n≥6 (OME_{≥6}) and also formaldehyde, methylal (OME₁), methanol and water,
introduction of the product mixture M_{R1} into a distillation unit D1 and distillative separation of the product mixture M_{R1} into a first fraction which contains methylal (OME₁), H₃C—O—(CH₂O)₂—CH₃ (OME₂), formaldehyde, methanol and water and leaves the distillation unit D1 as top stream KS_{D1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the distillation unit D1 as bottom stream SS_{D1},
mixing of the top stream KS_{D1} with a methanol-containing stream S_{MeOH} to obtain a mixture M1,
reaction of the mixture M1 in at least one reaction zone RZ of a reactive distillation unit RD2 in the presence of a catalyst, wherein H₃C—O—(CH₂O)₂—CH₃ (OME₂) reacts to give methylal (OME₁) and formaldehyde, and formaldehyde and methanol react to give methylal (OME₁); and distillative separation into a first fraction which contains methylal (OME₁) and leaves the reactive distillation unit RD2 as top stream KS_{RD2}, and a second fraction which contains water and leaves the reactive distillation unit RD2 as bottom stream SS_{RD2},
introduction of the bottom stream SS_{D1} into a distillation unit D3 and distillative separation of the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) present in the bottom stream SS_{D1} into a fraction which leaves the distillation unit D3 as top stream KS_{D3}, and a fraction which leaves the distillation unit as bottom stream SS_{D3}.

Alternatively, the object is achieved according to a second independent embodiment by a process for preparing polyoxymethylene dimethyl ethers, comprising the following steps:
mixing of a stream S_{FA} containing a water-containing formaldehyde source with a stream S_{OME1} containing methylal, to obtain a reactant mixture M_{Reactant},
reaction of the reactant mixture M_{Reactant} in a reactor R1 to obtain a product mixture M_{R1} containing polyoxymethylene dimethyl ethers of the formulae H₃C—O—(CH₂O)₂—CH₃ (OME₂), H₃C—O—(CH₂O)₃₋₅—CH₃ (OME₃₋₅) and H₃C—O—(CH₂O)ₙ—CH₃ with n≥6 (OME_{≥6}) and also formaldehyde, methylal (OME₁), methanol and water,
mixing of the product mixture M_{R1} with a methanol-containing stream S_{MeOH} to obtain a mixture M1,
reaction of the mixture M1 in at least one reaction zone RZ of a reactive distillation unit RD1 in the presence of a catalyst, wherein H₃C—O—(CH₂O)₂—CH₃ (OME₂) reacts to give methylal (OME₁) and formaldehyde, and formaldehyde and methanol react to give methylal (OME₁); and distillative separation into a first fraction which contains water, methanol, formaldehyde, methylal (OME₁) and H₃C—O—(CH₂O)₂—CH₃ (OME₂) and leaves the reactive distillation unit RD1 as top stream KS_{RD1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the reactive distillation unit RD1 as bottom stream SS_{RD1},
introduction of the top stream KS_{RD1} into a distillation unit D2 and distillative separation into a first fraction which contains methylal and leaves the distillation unit D2 as top stream KS_{D2}, and a second fraction which contains water and leaves the distillation unit D2 as bottom stream SS_{D2},
introduction of the bottom stream SS_{RD1} into a distillation unit D3 and distillative separation of the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) present in the bottom stream SS_{RD1} into a fraction which leaves the distillation unit D3 as top stream KS_{D3}, and a fraction which leaves the distillation unit as bottom stream SS_{D3}.

As will be described in more detail below, the processes according to the invention enable effective distillative removal of the water and thus address one of the main challenges with the preparation process of polyoxymethylene ethers. The formaldehyde present in the mixture M1 and the formaldehyde formed during the reactions of OME₂ to give methylal and formaldehyde is chemically bound in the form of methylal by the methanol present in the top stream KS_{D1} (first independent embodiment of the invention) or the methanol present in the product mixture M_{R1} (second independent embodiment of the invention) and the methanol added in an externally defined manner via the methanol-containing stream S_{MeOH}. By way of the external methanol stream S_{MeOH}, the proportion of formaldehyde in the bottom stream SS_{RD2} (first independent embodiment of the invention) or in the top stream KS_{RD1} and in the bottom stream SS_{D2} (second independent embodiment of the invention) can therefore be adjusted in a controlled manner (for example essentially completely removed). The use of additional water removal units is not necessary.

The water-containing formaldehyde source used in both independent embodiments of the present invention is preferably an aqueous formaldehyde solution, in particular a concentrated aqueous formaldehyde solution having a formaldehyde content of at least 70% by weight, more preferably at least 80% by weight, more preferably still at least 90% by weight. Such aqueous formaldehyde solutions are commercially available or can be prepared by known methods, for example from an aqueous formaldehyde-containing starting solution which passes through a concentrator unit (for example one or more thin-film evaporators) and is thus converted into a concentrated aqueous formaldehyde solution. The preparation of a concentrated aqueous formaldehyde solution is described for example in WO 03/040075 A2, EP 1 688 168 A1, DE 103 09 289 A1 or DE 103 09 286 A1.

The formaldehyde- and water-containing stream S_{FA} thus preferably has a content of formaldehyde of at least 70% by weight, more preferably at least 80% by weight, more preferably still at least 90% by weight, for example in the range from 70-97% by weight, more preferably 80-95% by weight or 90-95% by weight.

As is known to those skilled in the art, in an aqueous formaldehyde solution the monomeric formaldehyde CH₂O is present alongside the monomeric hydrate thereof methylene glycol (HO—(CH₂O)₁—H) and the oligomeric hydrates thereof, also referred to as polyoxymethylene glycols (HO—(CH₂O)ₙ—H with n≥2). The formaldehyde content of the aqueous formaldehyde solution relates to the total amount of monomeric formaldehyde CH₂O, monomeric formaldehyde hydrate (i.e. methylene glycol (HO—(CH₂O)₁—H)) and oligomeric formaldehyde hydrates (i.e. polyoxymethylene glycols (HO—(CH₂O)ₙ—H with n≥2)).

Both in the first and in the second embodiment of the process according to the invention, the formaldehyde- and water-containing stream S_{FA} is mixed with a stream S_{OME1} containing methylal (H₃C—O—(CH₂O)₁—CH₃, also referred to as dimethoxymethane or OME₁) to obtain a reactant mixture M_{Reactant}. As will be described in more detail below, the methylal-containing stream S_{OME1} is preferably the top stream KS_{RD2} drawn off from the reactive distillation unit RD2 (first embodiment of the invention) or the top stream KS_{D2} drawn off from the distillation unit D2 (second embodiment of the invention), which has been recycled for mixing with the water-containing formaldehyde source S_{FA}. A different methylal source may be used for starting up the process according to the invention. Preferably, the methylal-containing stream SO_{ME}1 has a content of methylal of at least 70% by weight, more preferably at least 90% by weight. Optionally, the methylal-containing stream S_{OME1} may contain further components, such as for example methanol. However, the content of methanol in S_{OME1} is preferably <10% by weight.

The formaldehyde- and water-containing stream S_{FA} is preferably mixed with the methylal-containing stream S_{OME1} outside of the reactor R1 and the reactant mixture M_{Reactant} is then introduced into the reactor R1. Alternatively, it is however also possible for the streams S_{FA} and S_{OME1} not to be mixed with one another until in reactor R1.

Besides formaldehyde, methylal and water, the reactant mixture M_{Reactant} may optionally also contain further components such as, for example, methanol or a number of longer-chain polyoxymethylene dimethyl ethers of the general formula H₃C—O—(CH₂O)ₙ—CH₃ with n≥6 (OME_{≥6}) The longer-chain polyoxymethylene dimethyl ethers have for example been drawn off from distillation unit D3 as bottom stream SS_{D3} and recycled.

The molar ratio of methylal to formaldehyde in the reactant mixture M_{Reactant} is for example in the range from 0.3 to 2.0, more preferably 0.5 to 1.5. However, lower or higher values for the molar methylal/formaldehyde ratio may also be selected. The preferred molar ratio depends on the formaldehyde content in the formaldehyde- and water-containing stream S_{FA} and on the methylal content of the methylal-containing stream S_{OME1}.

Both in the first and in the second embodiment of the process according to the invention, the reactant mixture M_{Reactant} is reacted in a reactor R1 to obtain a product mixture M_{R1} containing polyoxymethylene dimethyl ethers of the formulae H₃C—O—(CH₂O)₂—CH₃ (OME₂), H₃C—O—(CH₂O)₃₋₅—CH₃ (OME₃₋₅) and H₃C—O—(CH₂O)ₙ—CH₃ with n≥6 (OME_{≥6}) and also formaldehyde, methylal (OME₁), methanol and water. Suitable conditions for the formation of polyoxymethylene dimethyl ethers OME_{≥2} from the reactants formaldehyde and methylal (OME₁) are known to those skilled in the art. The reaction is preferably effected in the presence of an acidic catalyst. Solid catalysts or else liquid acids may be used. By way of example, the following catalysts may be cited: An ion-exchange resin having acidic groups (i.e. a cation-exchange resin), a zeolite, an aluminosilicate, an aluminum oxide, a transition metal oxide (which is optionally on a support material), a graphene oxide, a mineral acid (e.g. sulfuric acid), an organic acid (e.g. a sulfonic acid), an acidic ionic liquid, an oxonium salt (e.g. a trimethyloxonium salt). The reactor R1 is operated for example at a pressure of 1-10 bar and a temperature of 50-120° C. The reactor R1 is for example a fixed-bed reactor. However, within the context of the present invention, other reactor types may also be used for the reaction of the reactant mixture M_{Reactant}. Besides OME₂, OME₃₋₅, OME_{≥6}, formaldehyde, OME₁, methanol and water, the product mixture M_{R1} may optionally also contain further components, for example hemiacetals of the formula HO—(CH₂O)ₙ—CH₃ with n≥1, glycols of the formula HO—(CH₂O)ₙ—H with n≥1, trioxane and/or methyl formate.

According to the first independent embodiment of the process according to the invention, in a distillation unit D1 the product mixture M_{R1} is distillatively separated into a first fraction which contains methylal (OME₁), H₃C—O—(CH₂O)₂—CH₃ (OME₂), formaldehyde, methanol and water and leaves the distillation unit D1 as top stream KS_{D1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the distillation unit D1 as bottom stream SS_{D1}, Further components that may optionally be present in the top stream KS_{D1} are for example hemiacetals of the formula HO—(CH₂O)ₙ—CH₃ n≥2, glycols of the formula HO—(CH₂O)ₙ—H with n≥1, OME₃, trioxane and/or methyl formate. The top stream KS_{D1} preferably does not contain any OME_{≥4}, still more preferably does not contain any OME_{≥3}. Distillation unit D1 is preferably catalyst-free (in particular free from acidic catalysts). The distillation unit D1 is preferably not a reactive distillation unit.

The distillation unit D1 with respect to the distillative separation of the polyoxymethylene dimethyl ethers OME₂, OME₃₋₅ and OME_{≥6} is therefore designed so that OME₁₋₂ are drawn off in the top stream and OME_{≥3} are drawn off in the bottom stream from the distillation unit D1. Those skilled in the art can ascertain the conditions suitable for this by taking into account general specialist knowledge. The distillation unit D1 is for example a distillation column. The distillation unit typically contains internals for the distillative separation, in particular trays, random packings or structured packings, as are generally known to those skilled in the art. The distillation unit D1 is for example operated at a pressure of 1-15 bar and a temperature of 60-250° C. In order, if needed, to promote the reverse reaction of longer-chain hemiacetals and glycols to formaldehyde, methanol, water, HO—(CH₂O)₁—CH₃ (HF₁) and HO—(CH₂O)₁—H (MG₁) according to the abovementioned reaction equations 1-4, it may be advantageous to choose a relatively long residence time in the distillation unit D1. Measures that can optionally be used to prolong the residence time are known to those skilled in the art. In this context, reference may for example be made to the measures described in paragraph [0068] of DE 10 2016 222 657 A1. For example, the distillation unit D1 contains hold-up packings (as are described for example in EP 1 074 296 A1) or delay trays (e.g. Thormann® trays).

The top stream KS_{D1} drawn off from the distillation unit D1 in the first independent embodiment of the process according to the invention is mixed with a methanol-containing stream S_{MeOH} to obtain a mixture M1. Optionally, the methanol-containing stream S_{MeOH} may also contain further components (e.g. formaldehyde, water, OME₁, OME_{≥2} or trioxane). However, it is preferable for the methanol-containing stream S_{MeOH} to contain at least 80% by weight of methanol, more preferably at least 90% by weight of methanol.

In the first independent embodiment of the process according to the invention, the mixture M1 is reacted in at least one reaction zone RZ of a reactive distillation unit RD2 in the presence of a catalyst, wherein H₃C—O—(CH₂O)₂—CH₃ (OME₂) reacts to give methylal (OME₁) and formaldehyde, and formaldehyde and methanol react to give methylal (OME₁); and distillative separation is effected into a first fraction which contains methylal (OME₁) and leaves the reactive distillation unit RD2 as top stream KS_{RD2}, and a second fraction which contains water and leaves the reactive distillation unit RD2 as bottom stream SS_{RD2}.

The mixing of the top stream KS_{D1} drawn off from the distillation unit D1 with the methanol-containing stream S_{MeOH} preferably takes place outside of the reactive distillation unit RD2 and the resulting mixture M1 is then introduced into the reactive distillation unit RD2. However, in the context of the present invention it is also possible for the top stream KS_{D1} and the methanol-containing stream S_{MeOH} not to be mixed with one another until in the reactive distillation unit RD2.

The reactive distillation unit RD2 (e.g. a reactive distillation column) used in the first independent embodiment of the process according to the invention includes one or more reaction zones RZ and one or more distillative separation zones. The reaction zone RZ contains one or more catalysts, in particular an acidic catalyst (for example one or more acidic solid catalysts, for example an ion-exchange resin having acidic groups (i.e. a cation-exchange resin), a zeolite, an aluminosilicate, an aluminum oxide, a transition metal oxide (which is optionally on a support material) or a graphene oxide) for the reaction of OME₂ to give methylal (OME₁) and formaldehyde (see above reaction equation 7) and also the reaction of formaldehyde and methanol to give methylal (OME₁). The distillative separation zone for example contains internals for the distillative separation, in particular trays, random packings or structured packings, as are generally known to those skilled in the art. The catalyst may be immobilized in the reaction zones RZ of the reactive distillation unit RD2 in a manner known to those skilled in the art, for example as random dumped packings; in the form of catalyst-filled wire mesh spheres or as catalyst shaped bodies that are fitted to a tray in the reaction zone RZ. If the reactive distillation unit RD2 contains two or more reaction zones RZ, it may be preferable for a distillative separation zone to be present between each two reaction zones RZ.

In the catalyst-containing reaction zone RZ of the reactive distillation unit RD2, a chemical reaction of the mixture M1 is effected, wherein H₃C—O—(CH₂O)₂—CH₃ (OME₂) reacts to give methylal (OME₁) and formaldehyde, and formaldehyde and methanol react to give methylal (OME₁). In addition, in the reactive distillation unit RD2 a distillative separation is effected into a first fraction which contains methylal (OME₁) and optionally methanol and leaves the reactive distillation unit RD2 as top stream KS_{RD2}, and a second fraction which contains water and optionally excess methanol, unconverted formaldehyde, hemiacetals of the formula HO—(CH₂O)ₙ—CH₃ n≥1 and/or glycols of the formula HO—(CH₂O)ₙ—H with n≥1 and leaves the reactive distillation unit RD2 as bottom stream SS_{RD2}.

The reactive distillation unit RD2 makes it possible to
obtain a top stream KS_{RD2} which essentially contains methylal and optionally a small amount of methanol and thus can be recycled as methylal source for mixing with the water-containing formaldehyde source S_{FA} and
effectively remove the water via the bottom stream SS_{RD2}, which contains water and optionally excess methanol, unconverted formaldehyde, hemiacetals of the formula HO—(CH₂O)ₙ—CH₃ n≥1 and/or glycols of the formula HO—(CH₂O)ₙ—H with n≥1.

As mentioned above, formaldehyde and methanol react to give methylal (OME₁) in the catalyst-containing reaction zone RZ of the reactive distillation unit RD2. The molar ratio of formaldehyde to methanol in the mixture M1 can be used to control whether (i) the formaldehyde is to a large part or even completely converted to OME₁ and an unconverted residue of methanol remains or (ii) an unconverted residue of formaldehyde remains. In variant (i) the bottom stream SS_{RD2}, besides the water, preferably also contains methanol and optionally a relatively small amount of formaldehyde, whereas in variant (ii) the bottom stream SS_{RD2}, besides the water, preferably also contains formaldehyde and optionally a relatively small amount of methanol.

In variant (i) the water-containing bottom stream SS_{RD2} contains methanol for example in a proportion of 0% by weight to 80% by weight, more preferably 0% by weight to 30% by weight. The total proportion of water and methanol in the bottom stream SS_{RD2} is preferably more than 95% by weight. In addition, proportions of formaldehyde, H₃C—O—(CH₂O)₂—CH₃ (OME₂) and/or methylal (OME₁) may optionally also be present in the bottom stream SS_{RD2}, these preferably amounting in total to a proportion of <5% by weight, particularly preferably a proportion of <1% by weight.

In variant (ii) the water-containing bottom stream SS_{RD2} contains formaldehyde for example in a proportion of 0% by weight to 60% by weight, more preferably 25% by weight to 55% by weight. The total proportion of water and formaldehyde in the bottom stream SS_{RD2} is preferably more than 80% by weight, more preferably more than 95% by weight. In addition, proportions of unreacted MeOH, H₃C—O—(CH₂O)₂—CH₃ (OME₂) and/or methylal (OME₁) may optionally also be present in the bottom stream SS_{RD2}, these preferably amounting in total to a proportion of <20% by weight (with the proportion of OME₂ in the bottom stream SS_{RD2} preferably being less than 5% by weight), particularly preferably a proportion of <5% by weight (with the proportion of OME₂ in the bottom stream SS_{RD2} preferably being less than 2% by weight). For example, the water-containing bottom stream SS_{RD2} contains 25-55% by weight of formaldehyde, wherein the total proportion of water and formaldehyde in the bottom stream is more than 95% by weight and the proportion of OME₂ in the bottom stream SS_{RD2} is less than 2% by weight.

Suitable catalysts for the reaction of H₃C—O—(CH₂O)₂—CH₃ (OME₂) to give methylal (OME₁) and formaldehyde and for the reaction of formaldehyde and methanol to give methylal (OME₁) are known to those skilled in the art. Preference is given to an acidic catalyst (for example one or more acidic solid catalysts, for example an ion-exchange resin having acidic groups (i.e. a cation-exchange resin), a zeolite, an aluminosilicate, an aluminum oxide, a transition metal oxide (which is optionally on a support material) or a graphene oxide).

The reactive distillation unit RD2 is operated for example at a pressure in the range from 1-5 bar and a temperature in the range from 40-140° C.

For example, the mixture M1 is introduced into the reactive distillation unit RD2 in a region lying above the catalyst-containing reaction zone RZ. Optionally, a further catalyst-containing reaction zone RZ may be located above the region where the mixture M1 is introduced, and into this further reaction zone RZ is introduced a stream that contains predominantly (e.g. at least 80% by weight) formaldehyde, OME_{≥2} or trioxane or a mixture of at least two of these components. This may be advantageous for obtaining a top stream KS_{RD2} having a very low proportion of methanol.

Optionally, in addition to the top stream KS_{RD2} and the bottom stream SS_{RD2}, at least one further side stream, for example an MeOH-rich side stream having an MeOH content of at least 70% by weight, may be drawn off from the reactive distillation unit RD2. This side stream is drawn off for example from a region of the reactive distillation unit RD2 that lies between the reaction zone RZ and the draw-off region for the bottom stream SS_{RD2} (i.e. the bottom of the reactive distillation unit RD2).

In a preferred embodiment, at least a portion of the top stream KS_{RD2} drawn off from the reactive distillation unit RD2 is recycled and functions as methylal-containing stream S_{OME1}, which is mixed with the formaldehyde- and water-containing stream S_{FA} to obtain the reactant mixture M_{Reactant}. As already mentioned above, the two streams are preferably mixed upstream of the reactor R1 and the resulting reactant mixture M_{Reactant} is then introduced into the reactor R1. Alternatively, it is however also possible not to mix the two streams with one another until in reactor R1.

If there is still methanol present in the top stream KS_{RD2} drawn off from the reactive distillation unit RD2, it may for example be advantageous if the top stream KS_{RD2} during the recycling thereof passes through a distillation unit D4 in which the methanol is at least partially removed by distillation.

The top stream KS_{RD2} drawn off from the reactive distillation unit RD2 during the recycling thereof preferably passes through a mass flow divider in which a portion of the top stream KS_{RD2} is branched off. By using the mass flow divider, the ratio of methylal to formaldehyde in the reactant mixture M_{Reactant} can be regulated. This in turn assists with the establishment of a constant ratio of formaldehyde to methylal in the reactant mixture M_{Reactant} and the regulation of the proportions of the longer-chain polyoxymethylene dimethyl ethers OME_{≥3} in the final product. The methylal branched off in the mass flow divider for its part constitutes a potentially interesting starting material for other processes and can be stored until further use. Mass flow dividers with which product streams can be split into two or more substreams are known to those skilled in the art.

The bottom stream SS_{D1} drawn off from the distillation unit D1 in the first independent embodiment of the process according to the invention is introduced into a distillation unit D3. As mentioned above, the bottom stream SS_{D1} contains polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}). In the distillation unit D3, these polyoxymethylene dimethyl ethers are distillatively separated into a fraction which leaves the distillation unit D3 as top stream KS_{D3}, and a fraction which leaves the distillation unit as bottom stream SS_{D3}. The conditions chosen for the distillative separation in the distillation unit D3 can be adapted to the desired product spectrum. For example, the distillation unit D3 is operated so that the top stream KS_{D3} drawn off from the distillation unit D3 contains H₃C—O—(CH₂O)₃₋₅—CH₃ and the bottom stream SS_{D3} contains polyoxymethylene dimethyl ethers of the formula H₃C—O—(CH₂O)ₙ—CH₃ with n≥6. The distillation unit D3 for example contains internals for the distillative separation, in particular trays, random packings or structured packings, as are generally known to those skilled in the art. The distillation unit D3 is operated for example at a pressure in the range from 0.05-3 bar and a temperature in the range from 60-220° C. If the distillation unit is operated at a reduced pressure (0.05 bar to <1.0 bar), it may possibly be advantageous to use internals which result in a low pressure gradient. Distillation unit D3 is preferably catalyst-free (in particular free from acidic catalysts). The distillation unit D3 is preferably not a reactive distillation unit.

Optionally, at least a portion of the bottom stream SS_{D3} drawn off from the distillation unit D3 can be recycled and mixed with the formaldehyde- and water-containing stream S_{FA}.

As described above, the bottom stream SS_{RD2} drawn off from the reactive distillation unit RD2 may, besides water, optionally also contain formaldehyde (see the above-described variant (ii)). Since formaldehyde is one of the reactants of the process according to the invention, it may be advantageous in this case to recycle the formaldehyde-containing bottom stream SS_{RD2} and introduce it into a concentrator unit FC, wherein in the concentrator unit a portion of the water is removed and a stream leaves the concentrator unit which functions as stream S_{FA} and is mixed with the methylal-containing stream S_{OME1} to obtain the reactant mixture M_{Reactant}. Prior to being introduced into the concentrator unit FC, the recycled formaldehyde-containing bottom stream SS_{RD2} is preferably mixed with a formaldehyde-containing starting material (for example an aqueous formaldehyde solution having a formaldehyde content of at least 30% by weight, more preferably at least 50% by weight), to obtain a formaldehyde-containing mixture M2. The formaldehyde-containing mixture M2 is introduced into the concentrator unit FC and a portion of the water is removed in the concentrator unit FC to increase the concentration of formaldehyde. A stream is drawn off from the concentrator unit FC which functions as formaldehyde source S_{FA} and is mixed with the methylal-containing stream S_{OME1} to obtain the reactant mixture M_{Reactant}.

Suitable elements of a concentrator unit are known to those skilled in the art. For example, the concentrator unit contains one or more film evaporators. The film evaporator is, for example, a thin-film evaporator, a helical tube evaporator or a falling-film evaporator. With respect to suitable concentrator units for increasing the formaldehyde concentration in aqueous formaldehyde solutions, reference may be made to WO 03/040075 A2 and EP 1 688 168 A1.

In this constellation, there are three main process steps for the selective conversion of formaldehyde and methanol to longer-chain OMEs. The concentration of the formaldehyde source, the reaction of formaldehyde with the recycled methylal to give longer-chain OMEs and the reactive separation of the product mixture into a product stream which contains longer-chain OMEs, a product stream which predominantly contains water and optionally a product stream which predominantly contains methylal.

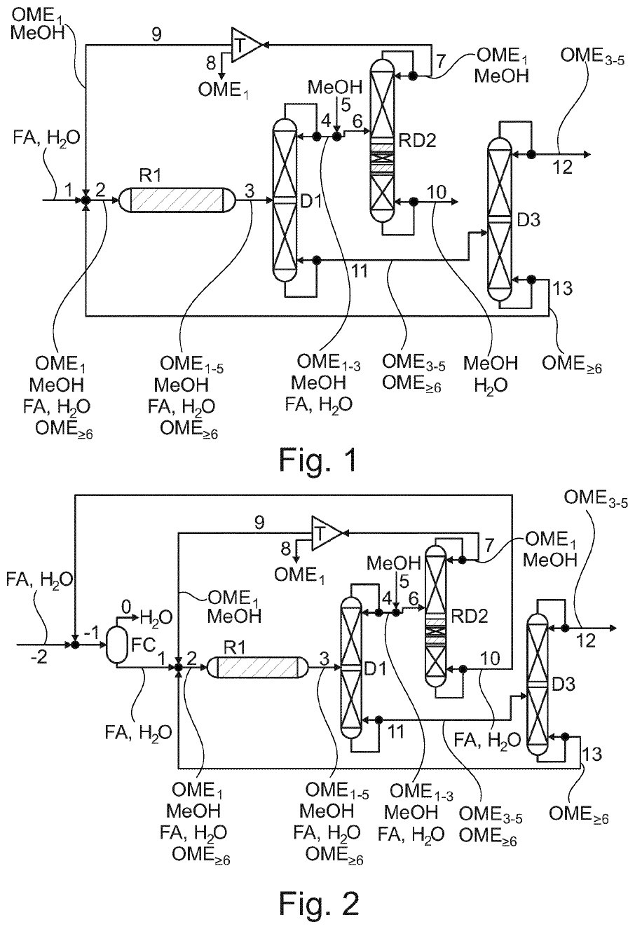

An exemplary configuration of the first independent embodiment of the present invention is described in more detail with reference to FIG. 1.

A formaldehyde- and water-containing stream S_{FA}, supplied via conduit 1, is mixed with a methylal-containing stream S_{OME1}, supplied via conduit 9. As will be described in more detail below, the methylal-containing stream S_{OME1} is the top stream KS_{RD2} that has been drawn off from the reactive distillation unit RD2 via conduit 7 and passes during the recycling thereof through a mass flow divider T. Optionally, the stream S_{OME1} may contain a small amount of methanol (<10% by weight). Optionally, longer-chain OMEs (e.g. OME_{≥6}) which as bottom stream SS_{D3} are drawn off from the distillation unit D3 via conduit 13 may be mixed with the streams S_{FA} und S_{OME1}.

By mixing the streams S_{FA} and S_{OME1} (and optionally SS_{D3}) a reactant mixture M_{Reactant} is obtained, which is introduced into a reactor R1, for example a fixed-bed reactor, via conduit 2. Formaldehyde and OME₁ react in reactor R1 to give OME₂, OME₃₋₅ and OME_{≥6}. A product mixture M_{R1} is obtained which contains OME₂, OME₃-s and OME_{≥6} and also OME₁, formaldehyde, methanol and water.

Via conduit 3, the product mixture M_{R1} is drawn off from reactor R1 and introduced into the distillation unit D1. In distillation unit D1, the product mixture M_{R1} is distillatively separated into a first fraction which contains methylal (OME₁), H₃C—O—(CH₂O)₂—CH₃ (OME₂), formaldehyde, methanol and water (and optionally OME₃) and leaves the distillation unit D1 via conduit 4 as top stream KS_{D1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the distillation unit D1 via conduit 11 as bottom stream SS_{D1}.

The top stream KS_{D1} drawn off from the distillation unit D1 via conduit 4 is mixed with a methanol-containing stream S_{MeOH}, supplied via conduit 5. The resulting mixture M1 is introduced via conduit 6 into a reactive distillation unit RD2. The molar ratio of methanol to formaldehyde in the mixture M1 is chosen so that the formaldehyde in the reactive distillation unit is completely converted to OME₁ and an unconverted residue of methanol remains.

The reactive distillation unit RD2 has catalyst-containing reaction zones in which OME₂ (and optionally OME₃, if the top stream KS_{D1} drawn off from the distillation unit D1 still contained a certain proportion of OME₃) is reacted to give methylal (OME₁) and formaldehyde, and formaldehyde and methanol are reacted to give methylal (OME₁). In addition, in the reactive distillation unit RD2 a distillative separation is effected into a first fraction which contains methylal (and optionally methanol) and leaves the reactive distillation unit RD2 via conduit 7 as top stream KS_{RD2}, and a second fraction which essentially contains water and methanol. This water-containing fraction leaves the reactive distillation unit RD2 via conduit 10 as bottom stream SS_{RD2}.

The top stream KS_{RD2} drawn off from the reactive distillation unit RD2 via conduit 7 is recycled and functions as methylal-containing stream S_{OME1}, which via conduit 9 is mixed with the formaldehyde- and water-containing stream S_{FA} (supplied via conduit 1) to obtain the reactant mixture M_{Reactant}. During the recycling thereof, the top stream KS_{RD2} passes through a mass flow divider T in which a portion of the top stream KS_{RD2} is branched off. By using the mass flow divider, the ratio of formaldehyde to methylal in the reactant mixture M_{Reactant} can be regulated. The methylal branched off in the mass flow divider for its part constitutes a potentially interesting starting material for other processes and can be stored until further use.

The bottom stream SS_{D1} drawn off from the distillation unit D1 via conduit 11 is introduced into a distillation unit D3. As already mentioned above, the bottom stream SS_{D1} contains OME₃₋₅ and OME_{≥6}. In the distillation unit D3, these polyoxymethylene dimethyl ethers are distillatively separated into a fraction which contains OME₃₋₅ and leaves the distillation unit D3 via conduit 12 as top stream KS_{D3}, and a fraction which contains OME_{≥6} and leaves the distillation unit D3 via conduit 13 as bottom stream SS_{D3}. Optionally, the bottom stream SS_{D3} may be recycled and mixed with the water-containing formaldehyde source S_{FA} (conduit 1).

A further exemplary configuration of the first independent embodiment of the process according to the invention is described in more detail with reference to FIG. 2. The process regime illustrated in FIG. 2 differs from the process regime illustrated in FIG. 1 as follows:
In the mixture M1 (obtained by mixing of the top stream KS_{D1} (conduit 4) with the methanol-containing stream S_{MeOH} (conduit 5)) the molar ratio of methanol to formaldehyde is chosen so that the methanol in the reactive distillation unit RD2 is to a very great extent converted into OME₁ and an unconverted residue of formaldehyde as constituent of the water-containing bottom stream SS_{RD2} is drawn off from the reactive distillation unit RD2 via conduit 10.
This bottom stream SS_{RD2}, which besides water also contains formaldehyde and optionally a small amount of methanol, is recycled and mixed with an aqueous formaldehyde solution supplied via conduit-2. The resulting mixture M2 is introduced via conduit-1 into the concentrator unit FC, which for example contains one or more thin-film evaporators, and a portion of the water is removed via conduit 0 in order to increase the concentration of formaldehyde. A stream is drawn off from the concentrator unit FC via conduit 1 and functions as formaldehyde source S_{FA}.

With respect to all further features of the exemplary configuration of the first independent embodiment of the present invention illustrated in FIG. 2, reference may be made to the above description relating to FIG. 1.

In an example of the process illustrated in FIG. 2, the reactor R1 was operated at 100° C. and 10 bar. The acidic catalyst used was Amberlyst® 46.

The reactant mixture M_{Reactant} supplied to the reactor R1 and the product mixture M_{R1} obtained in the reactor R1 had the compositions reported in table 1 below.

TABLE 1
Compositions of the reactant mixture M_{Reactant} and of the product
mixture M_{R1} obtained in the reactor R1

| Composition of reactant | Composition of product |
|---|---|
| mixture M_{Reactant} | mixture M_{R1} |
| 36% by weight of | 24% by weight of OME₁ |
| formaldehyde | 17% by weight of OME₂ |
| 4% by weight of water | 24% by weight of OME₃₋₅ |
| 60% by weight of OME₁ | 6% by weight of OME_{≥6} |
| | 19% by weight of |
| | formaldehyde |
| | 2% by weight of water |
| | 8% by weight of methanol |

After the distillative separation into a top stream KS_{D1} and a bottom stream SS_{D1} in the distillation unit D1, the top stream KS_{D1} was combined with a methanol-containing stream S_{MeOH} to obtain a mixture M1 and this mixture M1 was introduced into a reactive distillation unit RD2. The acidic catalyst used in the reactive distillation unit RD2 was Amberlyst® 46. The mixture M1 was introduced above the catalyst-containing reaction zone. During the distillation, a distillate temperature of 41° C. was established, which corresponds to the boiling temperature of the azeotropic mixture of OME₁ and methanol. A temperature of slightly above 100° C. was reached in the bottom of the reactive distillation unit RD2. RD2 was operated at ambient pressure.

The compositions of the mixture M1 introduced into the reactive distillation unit RD2 and of the top stream KS_{RD2} and bottom stream SS_{RD2} obtained in RD2 are reported in table 2 below.

TABLE 2
Compositions of the mixture M1 introduced into the reactive
distillation unit RD2 and of the top stream KS_{RD2} and bottom
stream SS_{RD2} obtained in RD2

| Composition of mixture | Composition of top | Composition of bottom |
|---|---|---|
| M1 | stream KS_{RD2} | stream SS_{RD2} |
| 18% by weight of | 94% by weight of | 44% by weight of |
| formaldehyde | OME1 | formaldehyde |
| 1% by weight of water | 6% by weight of | 56% by weight of water |
| 38% by weight of | methanol | |
| methanol | | |
| 23% by weight of OME₁ | | |
| 16% by weight of OME₂ | | |
| 3% by weight of OME₃ | | |

The bottom stream SS_{RD2} which consists essentially of water and formaldehyde can be recycled and thus become part of the water-containing formaldehyde starting source S_{FA}. The use of additional water removal units is not necessary.

The OME₂ present in the mixture M1 was essentially completely reacted so that bottom stream SS_{RD2} and top stream KS_{RD2} are essentially free from OME₂.

The second independent embodiment of the invention will be described in more detail below.

As already described above, both in the first and in the second independent embodiment of the present invention, the reactant mixture M_{Reactant} is first reacted in the reactor R1 to give the product mixture M_{R1}, which contains polyoxymethylene dimethyl ethers of the formulae H₃C—O—(CH₂O)₂—CH₃ (OME₂), H₃C—O—(CH₂O)₃₋₅—CH₃ (OME₃₋₅) and H₃C—O—(CH₂O)ₙ—CH₃ with n≥6 (OME_{≥6}) and also formaldehyde, methylal (OME₁), methanol and water.

In the second independent embodiment of the present invention, the product mixture M_{R1} obtained in the reactor R1 is mixed with a methanol-containing stream S_{MeOH}. The resulting mixture M1 is reacted in at least one reaction zone RZ of a reactive distillation unit RD1 in the presence of a catalyst, wherein H₃C—O—(CH₂O)₂—CH₃ (OME₂) reacts to give methylal (OME₁) and formaldehyde, and formaldehyde and methanol react to give methylal (OME₁); and distillative separation is effected into a first fraction which contains water, methanol, formaldehyde, methylal (OME₁) and H₃C—O—(CH₂O)₂—CH₃ (OME₂) and leaves the reactive distillation unit RD1 as top stream KS_{RD1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the reactive distillation unit RD1 as bottom stream SS_{RD1}, The mixing of the product mixture M_{R1} drawn off from the reactor R1 with the methanol-containing stream S_{MeOH} preferably takes place outside of the reactive distillation unit RD1 and the resulting mixture M1 is then introduced into the reactive distillation unit RD1. However, in the context of the present invention it is also possible for the product mixture M_{R1} and the methanol-containing stream S_{MeOH} not to be mixed with one another until in the reactive distillation unit RD1.

Optionally, the methanol-containing stream S_{MeOH} may also contain further components (e.g. formaldehyde, water, OME₁, OME_{≥2} or trioxane). However, it is preferable for the methanol-containing stream S_{MeOH} to contain at least 80% by weight of methanol, more preferably at least 90% by weight of methanol.

The reactive distillation unit RD1 (e.g. a reactive distillation column) used in the second independent embodiment of the process according to the invention includes one or more reaction zones RZ and one or more distillative separation zones. The reaction zone RZ contains one or more catalysts, in particular an acidic catalyst (for example one or more acidic solid catalysts, for example an ion-exchange resin having acidic groups (i.e. a cation-exchange resin), a zeolite, an aluminosilicate, an aluminum oxide, a transition metal oxide (which is optionally on a support material) or a graphene oxide) for the reaction of OME₂ to give methylal (OME₁) and formaldehyde (see above reaction equation 7) and also the reaction of formaldehyde and methanol to give methylal (OME₁). The distillative separation zone for example contains internals for the distillative separation, in particular trays, random packings or structured packings, as are generally known to those skilled in the art. The catalyst may be immobilized in the reaction zones RZ of the reactive distillation unit RD1 in a manner known to those skilled in the art, for example as random dumped packings; in the form of catalyst-filled wire mesh spheres or as catalyst shaped bodies that are fitted to a tray in the reaction zone RZ. If the reactive distillation unit RD1 contains two or more reaction zones RZ, it may be preferable for a distillative separation zone to be present between each two reaction zones RZ.

In the catalyst-containing reaction zone RZ of the reactive distillation unit RD1, a chemical reaction of the mixture M1 is effected, wherein H₃C—O—(CH₂O)₂—CH₃ (OME₂) reacts to give methylal (OME₁) and formaldehyde, and formaldehyde and methanol react to give methylal (OME₁). In addition, in the reactive distillation unit RD1 a distillative separation is effected into a first fraction which contains water, methanol, formaldehyde, methylal (OME₁) and H₃C—O—(CH₂O)₂—CH₃ (OME₂) and optionally hemiacetals of the formula HO—(CH₂O)ₙ—CH₃ n≥1 and/or glycols of the formula HO—(CH₂O)ₙ—H with n≥1 and leaves the reactive distillation unit RD1 as top stream KS_{RD1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the reactive distillation unit RD1 as bottom stream SS_{RD1}.

The following can be achieved by means of the reactive distillation unit RD1 and the distillation unit D2 downstream of the reactive distillation unit RD1 (and which will be described in more detail below):
From the distillation unit D2, a top stream KS_{D2} can be drawn off which essentially contains methylal and optionally a small amount of methanol and thus can be recycled as methylal source for mixing with the water-containing formaldehyde source S_{FA}.
The water can be efficiently removed via the bottom stream SS_{D2} from the distillation unit D2.

Suitable catalysts for the reaction of H₃C—O—(CH₂O)₂—CH₃ (OME₂) to give methylal (OME₁) and formaldehyde and for the reaction of formaldehyde and methanol to give methylal (OME₁) in the reaction zones RZ of the reactive distillation unit RD1 are known to those skilled in the art. Preference is given to an acidic catalyst (for example one or more acidic solid catalysts, for example an ion-exchange resin having acidic groups (i.e. a cation-exchange resin), a zeolite, an aluminosilicate, an aluminum oxide, a transition metal oxide (which is optionally on a support material) or a graphene oxide).

The reactive distillation unit RD1 is operated for example at a pressure in the range from 1-15 bar and a temperature in the range from 60-250° C.

Preferably, the mixture M1 is introduced into the reactive distillation unit RD1 in a region lying below the catalyst-containing reaction zone RZ. If the reactive distillation unit RD1 comprises a plurality of reaction zones RZ, it is preferable for the mixture M1 to be introduced into the reactive distillation unit RD1 in a region that lies below all of the reaction zones RZ present in RD1.

As mentioned above, formaldehyde and methanol react to give methylal (OME₁) in the catalyst-containing reaction zone RZ of the reactive distillation unit RD1. The molar ratio of formaldehyde to methanol in the mixture M1 can be used to control whether (i) the formaldehyde is to a large part or even completely converted to OME₁ and an unconverted residue of methanol remains or (ii) an unconverted residue of formaldehyde remains.

As already mentioned above, the top stream KS_{RD1} drawn off from the reactive distillation unit RD1 contains water, methanol, formaldehyde, methylal (OME₁) and H₃C—O—(CH₂O)₂—CH₃ (OME₂). Both in variant (i) and in variant (ii), the top stream KS_{RD1} typically has a very low proportion of OME₂, for example less than 5% by weight, more preferably less than 2% by weight. In variant (i), the top stream KS_{RD1} has a very low proportion of formaldehyde, for example less than 5% by weight. In variant (ii), the top stream KSRD1 has a very low proportion of methanol, for example less than 5% by weight.

The top stream KS_{RD1} drawn off from the reactive distillation unit is introduced into a distillation unit D2 and distillative separation is effected into a first fraction which contains methylal and leaves the distillation unit D2 as top stream KS_{D2}, and a second fraction which contains water and leaves the distillation unit D2 as bottom stream SS_{D2}.

The distillation unit D2 is for example catalyst-free (in particular free from acidic catalysts). Alternatively, it is also possible within the context of the present invention for the distillation unit D2 to be a reactive distillation unit which includes one or more reaction zones RZ and one or more distillative separation zones. The reaction zone RZ contains one or more acidic catalysts (for example one or more acidic solid catalysts, for example an ion-exchange resin having acidic groups (i.e. a cation-exchange resin), a zeolite, an aluminosilicate, an aluminum oxide, a transition metal oxide (which is optionally on a support material) or a graphene oxide).

The distillation unit D2 is operated for example at a pressure in the range from 1-5 bar and a temperature in the range from 40-140° C.

In the distillation unit D2 distillative separation is effected into a first fraction which contains methylal and leaves the distillation unit D2 as top stream KS_{D2}, and a second fraction which contains water and leaves the distillation unit D2 as bottom stream SS_{D2}.

The proportion of OME₂ in the bottom stream SS_{D2} is typically very low, for example less than 5% by weight, preferably less than 2% by weight, more preferably still less than 1% by weight.

If the variant (i) described above was used (i.e. excess of methanol, so that the top stream KS_{RD1} drawn off from the reactive distillation unit RD1 had a very low proportion of formaldehyde), the bottom stream SS_{D2} for example contains methanol in a proportion of 0% by weight to 80% by weight, more preferably 0% by weight to 30% by weight. The total proportion of water and methanol in the bottom stream SS_{D2} is preferably more than 95% by weight. The bottom stream SS_{D2} optionally also contains formaldehyde, H₃C—O—(CH₂O)₂—CH₃ (OME₂) and/or methylal (OME₁), these preferably amounting in total to a proportion of <5% by weight, particularly preferably a proportion of <1% by weight.

If the variant (ii) described above was used (i.e. a greater proportion of unconverted formaldehyde in the top stream KS_{RD1} drawn off from the reactive distillation unit RD1), the bottom stream SS_{D2} for example contains formaldehyde in a proportion of ≥0% by weight to 60% by weight, more preferably 25% by weight to 55% by weight. The total proportion of water and formaldehyde in the bottom stream SS_{D2} is preferably more than 80% by weight, more preferably more than 95% by weight. The bottom stream SS_{RD2} optionally also contains MeOH, H₃C—O—(CH₂O)₂—CH₃ (OME₂) and/or methylal (OME₁), these preferably amounting in total to a proportion of <20% by weight (with the proportion of OME₂ in the bottom stream SS_{D2} preferably being less than 5% by weight), particularly preferably a proportion of <5% by weight (with the proportion of OME₂ in the bottom stream SS_{D2} preferably being less than 2% by weight). For example, the water-containing bottom stream SS_{D2} contains 25-55% by weight of formaldehyde, wherein the total proportion of water and formaldehyde in the bottom stream is more than 95% by weight and the proportion of OME₂ in the bottom stream SS_{D2} is less than 2% by weight.

In a preferred embodiment, at least a portion of the top stream KS_{D2} drawn off from the distillation unit D2 is recycled and functions as methylal-containing stream S_{OME1}, which is mixed with the formaldehyde- and water-containing stream S_{FA} to obtain the reactant mixture M_{Reactant}. As already mentioned above, the two streams are preferably mixed upstream of the reactor R1 and the resulting reactant mixture M_{Reactant} is then introduced into the reactor R1. Alternatively, it is however also possible not to mix the two streams with one another until in reactor R1.

If there is still methanol present in the top stream KS_{D2} drawn off from the distillation unit D2, it may for example be advantageous if the top stream KS_{D2} during the recycling thereof passes through a distillation unit D4 in which the methanol is at least partially removed by distillation.

The top stream KS_{D2} drawn off from the distillation unit D2 during the recycling thereof preferably passes through a mass flow divider in which a portion of the top stream KS_{D2} is branched off. By using the mass flow divider, the ratio of methylal to formaldehyde in the reactant mixture M_{Reactant} can be regulated. This in turn assists with the establishment of a constant ratio of formaldehyde to methylal in the reactant mixture M_{Reactant} and the regulation of the proportions of the longer-chain polyoxymethylene dimethyl ethers OME_{≥3} in the final product. The methylal branched off in the mass flow divider for its part constitutes a potentially interesting starting material for other processes and can be stored until further use. Mass flow dividers with which product streams can be split into two or more substreams are known to those skilled in the art.

The bottom stream SS_{RD1} drawn off from the reactive distillation unit RD1 in the second independent embodiment of the process according to the invention is introduced into a distillation unit D3. As mentioned above, the bottom stream SS_{RD1} contains polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}). In the distillation unit D3, these polyoxymethylene dimethyl ethers are distillatively separated into a fraction which leaves the distillation unit D3 as top stream KS_{D3}, and a fraction which leaves the distillation unit as bottom stream SS_{D3}. The conditions chosen for the distillative separation in the distillation unit D3 can be adapted to the desired product spectrum. For example, the distillation unit D3 is operated so that the top stream KS_{D3} drawn off from the distillation unit D3 contains H₃C—O—(CH₂O)₃₋₅—CH₃ and the bottom stream SS_{D3} contains polyoxymethylene dimethyl ethers of the formula H₃C—O—(CH₂O)ₙ—CH₃ with n≥6. The distillation unit D3 for example contains internals for the distillative separation, in particular trays, random packings or structured packings, as are generally known to those skilled in the art. The distillation unit D3 is operated for example at a pressure in the range from 0.05-3 bar and a temperature in the range from 60-220° C. If the distillation unit is operated at a reduced pressure (0.05 bar to <1.0 bar), it may possibly be advantageous to use internals which result in a low pressure gradient. Distillation unit D3 is preferably catalyst-free (in particular free from acidic catalysts). The distillation unit D3 is preferably not a reactive distillation unit.

As in the first independent embodiment, in the second independent embodiment of the present invention at least a portion of the bottom stream SS_{D3} drawn off from the distillation unit D3 may also optionally be recycled and mixed with the formaldehyde- and water-containing stream S_{FA}.

As described above, the bottom stream SS_{D2} drawn off from the distillation unit D2 may, besides water, optionally also contain formaldehyde (see the above-described variant (ii)). Since formaldehyde is one of the reactants of the process according to the invention, it may be advantageous in this case to recycle the formaldehyde-containing bottom stream SS_{D2} and introduce it into a concentrator unit FC, wherein in the concentrator unit a portion of the water is removed and a stream leaves the concentrator unit which functions as stream S_{FA} and is mixed with the methylal-containing stream S_{OME1} to obtain the reactant mixture M_{Reactant}. Prior to being introduced into the concentrator unit FC, the recycled formaldehyde-containing bottom stream SS_{D2} is preferably mixed with a formaldehyde-containing starting material (for example an aqueous formaldehyde solution having a formaldehyde content of at least 30% by weight, more preferably at least 50% by weight), to obtain a formaldehyde-containing mixture M2. The formaldehyde-containing mixture M2 is introduced into the concentrator unit FC and a portion of the water is removed in the concentrator unit FC to increase the concentration of formaldehyde. A stream is drawn off from the concentrator unit FC which functions as formaldehyde source S_{FA} and is mixed with the methylal-containing stream S_{OME1} to obtain the reactant mixture M_{Reactant}.

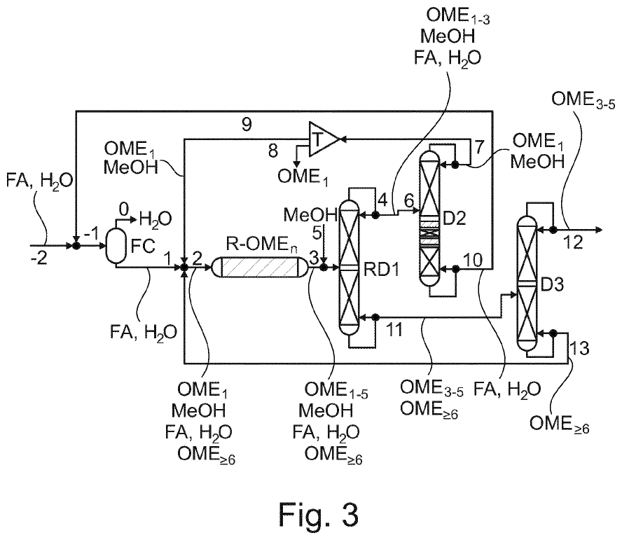

An exemplary configuration of the second independent embodiment of the present invention is described in more detail with reference to FIG. 3.

An aqueous formaldehyde solution, supplied via conduit-2, and a bottom stream SS_{D2} recycled from the distillation unit D2 and also containing, besides water, formaldehyde and optionally methanol, are mixed. The resulting mixture M2 is introduced via conduit-1 into the concentrator unit FC, which for example contains one or more thin-film evaporators, and a portion of the water is removed via conduit 0 in order to increase the concentration of formaldehyde. A stream is drawn off from the concentrator unit FC via conduit 1 and functions as formaldehyde source S_{FA}.

The formaldehyde source S_{FA}, supplied via conduit 1, is mixed with a methylal-containing stream S_{OME1}, supplied via conduit 9. The methylal-containing stream S_{OME1} is the top stream KS_{D2} that has been drawn off from the distillation unit D2 via conduit 7 and passes during the recycling thereof through a mass flow divider T. Optionally, the stream S_{OME1} may contain a small amount of methanol (<10% by weight). Optionally, longer-chain OMEs (e.g. OME_{≥6}) which as bottom stream SS_{D3} are drawn off from the distillation unit D3 via conduit 13 may be mixed with the streams S_{FA} und S_{OME1}.

By mixing the streams S_{FA} and S_{OME1} (and optionally SS_{D3}) a reactant mixture M_{Reactant} is obtained, which is introduced into a reactor R1, for example a fixed-bed reactor, via conduit 2. Formaldehyde and OME₁ react in the reactor R1 in the presence of an acidic catalyst to give OME₂, OME₃₋₅ and OME_{≥6}. A product mixture M_{R1} is obtained which contains OME₂, OME₃₋₅ and OME_{≥6} and also OME₁, formaldehyde, methanol and water.

Via conduit 3, the product mixture M_{R1} is drawn off from the reactor R1 and mixed with a methanol-containing stream S_{MeOH} supplied via conduit 5. The resulting mixture M1 is introduced into a reactive distillation unit RD1. The reactive distillation unit RD1 includes a plurality of reaction zones, each containing an acidic catalyst, and distillative separation zones. The mixture M1 is introduced into the reactive distillation unit RD1 below the catalyst-containing reaction zones RZ. The molar ratio of methanol to formaldehyde in the mixture M1 is chosen so that the methanol in the reactive distillation unit RD1 is to a very great extent converted into OME₁ and the top stream SS_{RD1} drawn off from RD1 therefore has a relatively low proportion of methanol.

In the catalyst-containing reaction zones of the reactive distillation unit RD1, OME₂ is reacted to give methylal (OME₁) and formaldehyde, and formaldehyde and methanol are also reacted to give methylal (OME₁). In addition, in the reactive distillation unit RD1 a distillative separation is effected into a first fraction which contains water, methanol, formaldehyde, methylal (OME₁) and H₃C—O—(CH₂O)₂—CH₃ (OME₂) and leaves the reactive distillation unit RD1 as top stream KS_{RD1}, and a second fraction which contains the polyoxymethylene dimethyl ethers of the formulae H₃C—O—(CH₂O)₃₋₅—CH₃ (OME₃₋₅) and H₃C—O—(CH₂O)ₙ—CH₃ with n≥6 (OME_{≥6}) and leaves the reactive distillation unit RD1 as bottom stream SS_{RD1}.

Via conduit 6, the top stream KS_{RD1} drawn off from RD1 is introduced into a catalyst-free distillation unit D2. In this distillation unit D2 distillative separation is effected into a first fraction which contains methylal and leaves the distillation unit D2 as top stream KS_{D2}, and a second fraction which contains water and formaldehyde and leaves the distillation unit D2 as bottom stream SS_{D2}.

The top stream KS_{D2} drawn off from the distillation unit D2 via conduit 7 is recycled and functions as methylal-containing stream S_{OME1}, which via conduit 9 is mixed with the aqueous formaldehyde solution (supplied via conduit-2). During the recycling thereof, the top stream KS_{D2} passes through a mass flow divider T in which a portion of the top stream KS_{D2} is branched off. By using the mass flow divider, the ratio of formaldehyde to methylal in the reactant mixture M_{Reactant} can be regulated. The methylal branched off in the mass flow divider for its part constitutes a potentially interesting starting material for other processes and can be stored until further use.

The aqueous, formaldehyde-containing bottom stream SS_{D2} drawn off from the distillation unit D2 via conduit 10, which consists essentially of water and formaldehyde, is recycled and mixed with the aqueous formaldehyde starting solution supplied via conduit-2.

The bottom stream SS_{RD1} drawn off from the reactive distillation unit RD1 via conduit 11 is introduced into a distillation unit D3. The bottom stream SS_{D1} contains OME₃₋₅ and OME_{≥6}. In the distillation unit D3, these polyoxymethylene dimethyl ethers are distillatively separated into a fraction which contains OME₃₋₅ and leaves the distillation unit D3 via conduit 12 as top stream KS_{D3}, and a fraction which contains OME_{≥6} and leaves the distillation unit D3 via conduit 13 as bottom stream SS_{D3}. Optionally, the bottom stream SS_{D3} may be recycled and mixed with the water-containing formaldehyde source S_{FA} (conduit 1).

1. A process for preparing polyoxymethylene dimethyl ethers, comprising:
mixing of a stream S_{FA} containing a water-containing formaldehyde source with a stream S_{OME1} containing methylal, to obtain a reactant mixture M_{Reactant},
reacting the reactant mixture M_{Reactant} in a reactor R1 to obtain a product mixture M_{R1} containing polyoxymethylene dimethyl ethers of the formulae H₃C—O—(CH₂O)₂—CH₃, H₃C—O—(CH₂O)₃₋₅—CH₃ and
H₃C—O—(CH₂O)ₙ—CH₃ with n≥6 and also formaldehyde, methylal, methanol and water,
introducing the product mixture M_{R1} into a distillation unit D1 and distillative separation of the product mixture M_{R1} into a first fraction containing methylal, H₃C—O—(CH₂O)₂—CH₃, formaldehyde, methanol and water and leaves the distillation unit D1 as top stream KS_{D1}, and a second fraction containing the polyoxymethylene dimethyl ethers of the formulae H₃C—O—(CH₂O)₃₋₅—CH₃ and H₃C—O—(CH₂O)ₙ—CH₃ with n≥6 and leaves the distillation unit D1 as bottom stream SS_{D1},
mixing of the top stream KS_{D1} with a methanol-containing stream S_{MeOH} to obtain a mixture M1,
reacting the mixture M1 in at least one reaction zone RZ of a reactive distillation unit RD2 in the presence of a catalyst, wherein H₃C—O—(CH₂O)₂—CH₃ reacts to give methylal and formaldehyde, and formaldehyde and methanol react to give methylal; and distillative separation into a first fraction containing methylal (OME₁) and leaves the reactive distillation unit RD2 as top stream KS_{RD2}, and a second fraction containing water and leaves the reactive distillation unit RD2 as bottom stream SS_{RD2},
introducing the bottom stream SS_{D1} into a distillation unit D3 and distillative separation of the polyoxymethylene dimethyl ethers of the formulae H₃C—O—(CH₂O)₃₋₅—CH₃ and
H₃C—O—(CH₂O)ₙ—CH₃ with n≥6 present in the bottom stream SS_{D1} into a fraction which leaves the distillation unit D3 as top stream KS_{D3}, and a fraction which leaves the distillation unit as bottom stream SS_{D3}.

2. A process for preparing polyoxymethylene dimethyl ethers, comprising:
mixing of a stream S_{FA} containing a water-containing formaldehyde source with a stream S_{OME1} containing methylal, to obtain a reactant mixture M_{Reactant},
reacting the reactant mixture M_{Reactant} in a reactor R1 to obtain a product mixture M_{R1} containing polyoxymethylene dimethyl ethers of the formulae H₃C—O—(CH₂O)₂—CH₃, H₃C—O—(CH₂O)₃₋₅—CH₃ and
H₃C—O—(CH₂O)ₙ—CH₃ with n≥6 and also formaldehyde, methylal, methanol and water,
mixing of the product mixture M_{R1} with a methanol-containing stream S_{MeOH} to obtain a mixture M1,
reacting the mixture M1 in at least one reaction zone RZ of a reactive distillation unit RD1 in the presence of a catalyst, wherein H₃C—O—(CH₂O)₂—CH₃ reacts to give methylal and formaldehyde, and formaldehyde and methanol react to give methylal; and distillative separation into a first fraction containing water, methanol, formaldehyde, methylal and H₃C—O—(CH₂O)₂—CH₃ (OME₂) and leaves the reactive distillation unit RD1 as top stream KS_{RD1}, and a second fraction containing the polyoxymethylene dimethyl ethers of the formulae H₃C—O—(CH₂O)₃₋₅—CH₃ and
H₃C—O—(CH₂O)ₙ—CH₃ with n≥6 and leaves the reactive distillation unit RD1 as bottom stream SS_{RD1},
introducing the top stream KS_{RD1} into a distillation unit D2 and distillative separation into a first fraction containing methylal and leaves the distillation unit D2 as top stream KS_{D2}, and a second fraction containing water and leaves the distillation unit D2 as bottom stream SS_{D2},
introducing the bottom stream SS_{RD1} into a distillation unit D3 and distillative separation of the polyoxymethylene dimethyl ethers of formulae H₃C—O—(CH₂O)₃₋₅—CH₃ and
H₃C—O—(CH₂O)ₙ—CH₃ with n≥6 present in the bottom stream SS_{RD1} into a fraction which leaves the distillation unit D3 as top stream KS_{D3}, and a fraction which leaves the distillation unit as bottom stream SS_{D3}.

3. The process according to claim 1, wherein the water-containing formaldehyde source is an aqueous formaldehyde solution having a formaldehyde content of at least 70% by weight.

4. The process according to claim 1, wherein the reactant mixture M_{Reactant} is reacted in the reactor R1 in the presence of an acidic catalyst.

5. The process according to claim 1, wherein the catalyst present in the reaction zone RZ of the reactive distillation unit RD1 or RD2 is an acidic catalyst.

6. The process according to claim 1, wherein at least a portion of the top stream KS_{RD2} drawn off from the reactive distillation unit RD2 or of the top stream KS_{D2} drawn off from the distillation unit D2 is recycled and functions as methylal-containing stream S_{OME1}, which is mixed with the formaldehyde- and water-containing stream S_{FA} to obtain the reactant mixture M_{Reactant}.

7. The process according to claim 6, wherein the top stream KS_{RD2} or KS_{D2} during the recycling thereof passes through a mass flow divider in which a portion of the top stream KS_{RD2} or KS_{D2} is branched off.

8. The process according to claim 1, wherein the bottom stream SS_{RD2} drawn off from the reactive distillation unit RD2 or the bottom stream SS_{D2} drawn off from the distillation unit D2 also contains, besides water, methanol in a proportion of up to 80% by weight, wherein the total proportion of water and methanol in the bottom stream SS_{RD2} is more than 95% by weight.

9. The process according to claim 1, wherein the bottom stream SS_{RD2} drawn off from the reactive distillation unit RD2 or the bottom stream SS_{D2} drawn off from the distillation unit D2 also contains, besides water, formaldehyde in a proportion of up to 60% by weight and the total proportion of water and formaldehyde in the bottom stream SS_{RD2} is more than 80% by weight, more preferably more than 95% by weight.

10. The process according to claim 9, wherein the bottom stream SS_{RD2} drawn off from the reactive distillation unit RD2 or the bottom stream SS_{D2} drawn off from the distillation unit D2 is recycled into a concentrator unit FC, wherein in the concentrator unit FC a portion of the water is removed and a stream leaves the concentrator unit FC which functions as stream S_{FA} and is mixed with the methylal-containing stream S_{OME1} to obtain the reactant mixture M_{Reactant}.

11. The process according to claim 10, wherein the recycled formaldehyde-containing bottom stream SS_{RD2} or SS_{D2} prior to being introduced into the concentrator unit FC is mixed with a formaldehyde-containing starting material, in particular an aqueous formaldehyde solution, to obtain a formaldehyde-containing mixture M2, and the formaldehyde-containing mixture M2 is introduced into the concentrator unit FC.

12. The process according to claim 10, wherein the concentrator unit FC comprises at least one evaporator, in particular at least one thin-film evaporator.

13. The process according to claim 1, wherein the top stream KS_{D3} drawn off from the distillation unit D3 contains H₃C—O—(CH₂O)₃₋₅—CH₃ and the bottom stream SS_{D3} contains polyoxymethylene dimethyl ethers of the formula H₃C—O—(CH₂O)ₙ—CH₃ with n≥6.

14. The process according to claim 1, wherein the bottom stream SS_{D3} is recycled and mixed with the stream S_{FA} and/or the stream S_{OME1}.
